(19) United States
(12) Plant Patent Application Publication
(10) Pub. No.: US 2025/0107472 P1
(43) Pub. Date: Mar. 27, 2025

(54) Rose Plant Named 'Poulnap008'

(50) ;

(71) Applicant: Mogens Nyegaard Olesen, Fredensborg (DK)

(72) Inventor: Mogens Nyegaard Olesen, Fredensborg (DK)

(73) Assignee: Poulsen Roser A/S, Fredensborg (DK)

(21) Appl. No.: 18/831,186

(22) Filed: Sep. 5, 2024

(30) Foreign Application Priority Data
Sep. 22, 2023 (QZ) 20231974

(51) Int. Cl. A01H 6/74

(52) U.S. Cl. CPC A01H 6/749
USPC PLT/148

(57) ABSTRACT

A new garden rose plant of the floribunda class which has abundant, pink and apricot blend flowers with strong fragrance and attractive foliage. This new and distinct variety has shown to be uniform and stable in the resulting generations from asexual propagation.

This application claims priority to Plant Breeder's Rights Application Number 2023/1974, which was filed at the Community Plant Variety Rights Office in the European Union on Sep. 22, 2023, the contents of which are hereby incorporated by reference for all purposes.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2013 and the resulting seeds were planted in a controlled environment in Fredensborg, Denmark. The new variety, named ‘Poulnap008’, originated as a single seedling from the stated cross.

The new variety may be distinguished from its male pollen parent and female seed parent primarily by the following characteristics. The male pollen parent plant has larger flowers in less abundance than the claimed plant. The female seed parent plant has red flowers while the new variety has flowers with a pink and apricot color blend.

The objective of the hybridization of this rose variety was to create a new and distinct garden rose variety with unique qualities, such as:
1. Uniform and abundant pink and apricot blend flowers with perfume;
2. Vigorous, but compact growth when propagated on its own roots;
3. Exceptional disease resistance.

This combination of qualities is not present in previously available commercial cultivars of this type, known to the inventor, and distinguish ‘Poulnap008’ from all other varieties of which we are aware.

As part of the rose development program, Mogens N. Olesen germinated the seeds from the aforementioned hybridization during winter of 2013 and conducted evaluations on the resulting seedlings in a controlled environment in Fredensborg, Denmark. ‘Poulnap008’ was selected in the spring of 2014 by the inventor as a single plant from the progeny of the aforementioned hybridization.

Asexual reproduction of ‘Poulnap008’ by rooted cuttings was first done by Mogens N. Olesen in the nursery in Fredensborg, Denmark in July 2014. This initial and other subsequent asexual propagations conducted in controlled environments have demonstrated that the characteristics of ‘Poulnap008’ are true to type and are transmitted from one generation to the next.

### DESCRIPTION OF THE DRAWING

The accompanying color illustrations show as true as is reasonably possible to obtain in color photographs of this type, the typical characteristics of the buds, flowers, leaves, and stems, of ‘Poulnap008’. Specifically illustrated in

FIG. 1 of the drawings are open flowers at various stages of development viewed from above, a flower bud and one quarter opened flower viewed from the side, a fully open flower viewed from the side, and flower petals detached revealing reproductive flower parts. Specifically illustrated in

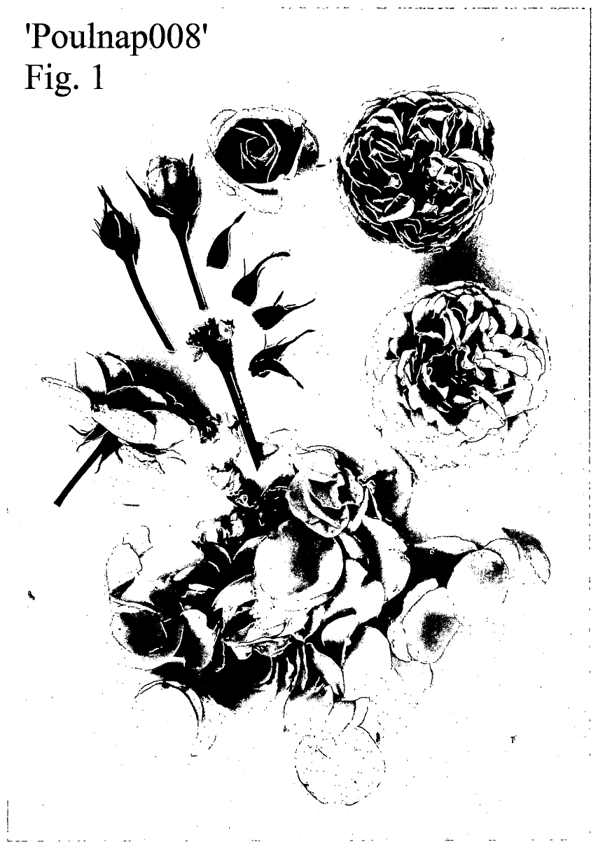

FIG. 2 of the drawings is a cluster of flower buds on the branch, juvenile growth, a bare stem exhibiting prickles, and a mature compound leaf. Plants shown are 15 months old.

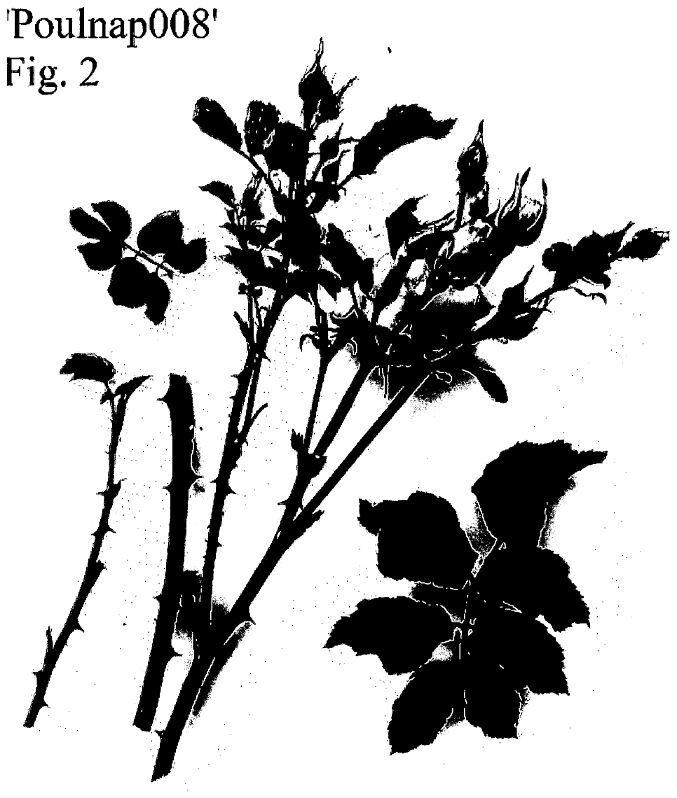

### DETAILED DESCRIPTION OF THE VARIETY

The following is a description of ‘Poulnap008’, as observed in its growth in an outdoor nursery in Odense Denmark. Observed plants are 15 months of age, and were grown on their own roots in 30 cm pots. Color references are made using the Royal Horticultural Society (London, England) Color Chart, 2001, except where common terms of color are used.

For a comparison, several physical characteristics of the rose variety ‘Poulren036’, U.S. Plant patent application Ser. No. 17/803,630 are compared to the claimed plant. While ‘Poulnap008’ has, in quantity, about 83 petals, ‘Poulren036’ has about 107 flower petals. The color of flower petal inner surface upon opening for that of ‘Poulnap008’ is described as Yellow Group 12C at the basal zone, middle and marginal zones are Red Group 38B with light shades of Yellow-Orange Group 19C throughout. The inner surface of flower petals upon opening for the comparison variety is Orange-Red Group 32C with a basal zone color Yellow-Orange Group 14B, with a basal zone of Yellow 11 A.

Flower and Flower Bud

| | |
|---|---|
| Blooming habit: | Continuous. |

Flower bud:

| | |
|---|---|
| Size: | Upon opening, 30 mm in length from base |
| | of receptacle to end of bud. Bud diameter |
| | is 13 mm. |
| Bud form: | Ucerolate. |
| Bud color: | As sepals divide petals are Yellow Group |
| | 8C. |

Sepal inner surface:

| | |
|---|---|
| Color: | Yellow-Green Group 145B with |
| | intonations of Greyed-Purple Group 183C. |
| Surface: | Lightly pubescent. |

Sepal outer surface:

| | |
|---|---|
| Color: | Yellow-Green Group 144A with |
| | intonations of Greyed-Red Group 183C at |
| | the apex. |
| Texture: | Smooth. |

Sepal Shape:

| | |
|---|---|
| Apex: | Cirrhose. |
| Base: | Flat at union with receptacle. |
| Sepal Margin: | Margins have weak foliaceous appendages |
| | on three of the five sepals. |
| Sepal size: | About 35 mm long, 11 mm wide. |

Receptacle:

| | |
|---|---|
| Texture: | Smooth. |
| Size: | 5 mm in height, 7 mm wide. |
| Color: | Yellow-Green Group 144A. |
| Shape: | Campanulate. |

Pedicel:

| Surface: | Smooth. | |
|---|---|---|
| Length: | 47 | mm. |

| | |
|---|---|
| Diameter: | 3.5 mm on average. |
| Color: | Yellow-Green Group 144A with light |
| | intonations of Greyed-Red Group 180A. |
| Strength: | Strong. |

Peduncle:

| | | |
|---|---|---|
| Length: | 5 to 12 | cm. |

| | |
|---|---|
| Diameter: | About 3 to 4 mm. |
| Color: | Yellow-Green Group 144A with |
| | intonations of Greyed-Purple Group 183C. |
| Texture: | Smooth and with large prickles the color |
| | of Greyed-Purple Group 183B. |
| Flower bud development: | Flower buds are borne in clusters of 3 to 5 |
| | flower buds on each peduncle. Normally |
| | stems have about 5 peduncles. |

Flower bloom:

| | |
|---|---|
| Fragrance: | Strong citrus like perfume. |
| Duration: | The blooms have a duration on the plant of |
| | approximately 18 days. Petals fall cleanly |
| | away from plant after flowers have fully |
| | matured. |
| Size: | Flower diameter is 75 to 85 mm when |
| | open. Flower depth is 35 mm. |
| Flower shape: | Quartered-rosette, very double, with many |
| | overlapping petals packed loosely into |
| | quarter sections. |
| Shape of flower, side view: | The upper portion is flat. The lower |
| | portion is concave. |
| Petalage: | Under normal conditions, flowers have |
| | about 83 petals. |
| General Tonality of Flower: | Open flowers are primarily Red Group |
| | 49A with intonations of Red Group 38A. |
| | After 10 days the color faces to Red Group |
| | 38D. |

Petal Color:
Upon opening, outer petals

| | |
|---|---|
| Upper surface: | At basal zone Yellow Group 12C. Middle |
| | and marginal zone Red Group 38B. Light |
| | shades of Yellow-Orange Group 19C |
| | throughout. |
| Lower surface: | At basal zone Yellow Group 10B. The |
| | middle zone is Yellow Group 19C. |
| | Marginal zone Red Group 49B and C |
| | blended. |

Upon opening, inner petals:

| | |
|---|---|
| Upper surface: | At basal zone Yellow Group 12C. Middle |
| | and marginal zone Red Group 38B. Light |
| | shades of Yellow-Orange Group 19C |
| | throughout. |
| Lower surface: | At basal zone Yellow Group 10B. The |
| | middle zone is Yellow Group 19C. |
| | Marginal zone Red Group 49B and C |
| | blended. |

After opening, outer petals:

| | |
|---|---|
| Upper surface: | Red Group 56B with basal intonations of |
| | Yellow Group 4D. |
| Lower surface: | At the basal zone, Yellow Group 4D. |
| | At the middle zone Yellow-Orange Group |
| | 19D. Marginal intonations are Red Group |
| | 56B. |

Petals:

| | |
|---|---|
| Petal Reflex: | Not reflexed. |
| Margin: | Entire and uniform. Light undulations. |
| Shape: | Rounded. |
| Apex shape: | Rounded. |
| Base shape: | Obtuse. |
| Size: | 45 mm (l) × 38 mm (w). |
| Texture: | Smooth. |
| Thickness: | Thin. |

Petaloids:

| | |
|---|---|
| Size: | 25 mm (l) by 11 mm (w). |
| Quantity: | About 12. |
| Shape: | Elliptical with an acute base and rounded |
| | apices. |
| Color: | Red Group 38C underneath. The upper |
| | side is Red Group 49C. Basal zone and |
| | streaking Yellow Group 8B. |

Reproductive Flower Parts:

| | |
|---|---|
| Pollen: | None observed. |

Anthers:

| | |
|---|---|
| Size: | 3 mm in length. |
| Color: | Yellow Group 11C. |
| Quantity: | 90 on average. |

Filaments:

| Color: | Yellow Group 12A. | |
|---|---|---|
| Length: | 5 | mm. |

Pistils:

| | | |
|---|---|---|
| Length: | 10 | mm. |

| | |
|---|---|
| Quantity: | 35 on average. |

Stigmas:

| | |
|---|---|
| Color: | Yellow Group 160C. |

Styles:

| | |
|---|---|
| Color: | Red-Purple Group 63B. |
| Location of stigmas: | Superior in location relative to the length |
| | of the filaments and the height of the |
| | anthers. |
| Hips: | None Observed. |

Plant

| | |
|---|---|
| Plant growth: | Upright, bushy. Plants are 50 cm in |
| | height, and 65 cm wide. |

Stems:

| | |
|---|---|
| Color of juvenile growth: | Yellow-Green Group 145A with strong |
| | intonations of Greyed-Red Group 178B. |
| Color of mature growth: | Yellow-Green Group 146B. |
| Length: | Canes are about 23 cm from the base of the |
| | plant to the flowering portion. |
| Diameter: | About 4 mm. |
| Internodes: | On mature canes about 45 mm between |
| | nodes. |

Surface Texture:

| | |
|---|---|
| Young wood: | Smooth. |
| Older wood: | Somewhat rough. |

Long Prickles:

| | |
|---|---|
| Incidence: | About 13 prickles per 10 cm of stem. |
| Size: | Average length of prickles on mature |
| | stems is 7 mm. |
| Shape: | Upper portion is concave. Lower portion |
| | is concave. |

Color:

| | |
|---|---|
| Juvenile prickles: | Greyed-Purple Group 184D and Yellow- |
| | Green Group 144C. |
| Mature prickles: | Greyed-Purple Group 183C. |

Plant foliage:

| | |
|---|---|
| Compound leaf: | 12.5 cm long × 10.5 cm wide. |
| Quantity: | 2 leaves per 10 cm of stem on average. |
| Leaf bearing angle to | 45 degrees. |
| the stem: | |

Color of juvenile foliage:

| | |
|---|---|
| Upper side: | Yellow-Green Group 144A shaded with |
| | Greyed-Red Group 180A. |
| Lower side: | Yellow-Green Group 144B. |

Color of mature foliage:

| | |
|---|---|
| Upper side: | Yellow-Green Group 147A. |
| Lower side: | Yellow-Green Group 147B. |

Plant leaves and leaflets:
Stipules:

| | |
|---|---|
| Size: | 20 mm long, 3 mm wide. |
| Quantity: | 2 per compound leaf. |
| Shape: | Linear, slightly broad based with outward |
| | extending apices. |
| Margins: | Finely serrated. |
| Color: | Yellow-Green Group 146A. |

Petiole:

| Length: | 44 mm average. | |
|---|---|---|
| Diameter: | 3 | mm. |

| | |
|---|---|
| Upper surface color: | Yellow-Green Group 144A. |
| Lower surface color: | Yellow-Green Group 144A. |

Rachis:

| | |
|---|---|
| Length: | 60 mm average. |
| Upper surface color: | Yellow-Green Group 144A. |
| Lower surface color: | Yellow-Green Group 144A. |

Leaflet:

| | |
|---|---|
| Quantity: | Normally 7 leaflets. |
| Margins: | Serrated. |
| Size: | Terminal leaflets are about |
| | 55 mm long, 48 mm wide. |
| Shape: | Generally elliptical. |
| Base: | Cordate or rounded. |
| Apex: | Mucronate. |
| Texture: | Smooth. |
| Thickness: | Average. |
| Arrangement: | Odd pinnate. |
| Venation: | Reticulate. |
| Glossiness: | Moderately glossy. |
| Disease resistance: | Above average resistance to powdery |
| | mildew Sphaerotheca pannosa var. rosae, |
| | downy mildew Peronospora sparsa, rust |
| | Phragmidium spp., black spot |
| | Diplocarpon rosae, and Botrytis cinerea |
| | under normal growing conditions. |
| Cold Hardiness: | The variety is tolerant to USDA Cold |
| | Hardiness Zone 6. |
| Heat Tolerance: | The variety has been found to be suitable |
| | for climate conditions found in the |
| | American Horticulture Society heat zone |
| | 7. |

I claim:

1. A new and distinct variety of rose plant named ‘Poulnap008’ substantially as described and illustrated herein.
